(19) United States
(12) Patent Application Publication
(10) Pub. No.: US 2025/0209618 A1
(43) Pub. Date: Jun. 26, 2025

(54) SETTING METHOD FOR MACHINE LEARNING MODEL, DIAGNOSIS SUPPORT SYSTEM, AND RECORDING MEDIUM

(71) Applicant: CASIO COMPUTER CO., LTD., Tokyo (JP)

(72) Inventor: Yuko ODA, Tokyo (JP)

(73) Assignee: CASIO COMPUTER CO., LTD., Tokyo (JP)

(21) Appl. No.: 18/982,435

(22) Filed: Dec. 16, 2024

(30) Foreign Application Priority Data
Dec. 21, 2023 (JP) 2023-215338

(51) Int. Cl. G06T 7/00; G16H 50/20

(52) U.S. Cl. CPC G06T 7/0012; G16H 50/20; G06T 2207/20081; G06T 2207/30096

(57) ABSTRACT

A diagnosis support system executes processing for detecting an input operation of specifying at least one of a sensitivity or a specificity of a machine learning model for medical diagnosis; processing for determining, based on the at least one of the sensitivity or the specificity specified by the detected input operation, a combination of a value of the sensitivity and a value of the specificity; and processing for setting a cut off value of the machine learning model corresponding to the determined combination.

### CROSS-REFERENCE TO RELATED APPLICATIONS

This application claims the benefit of Japanese Patent Application No. 2023-215338, filed on Dec. 21, 2023, the entire disclosure of which is incorporated by reference herein.

### FIELD OF THE INVENTION

This application relates generally to a setting method for a machine learning model, a diagnosis support system, and a recording medium.

### BACKGROUND OF THE INVENTION

The work of diagnosing is being supported by machine learning models in the medical field. For example, the device described in Patent Literature 1 (Unexamined Japanese Patent Application Publication No. 2022-038639) that outputs a disease name and, also, devices that identify positive or negative for a predetermined test are known in the medical field.

### SUMMARY OF THE INVENTION

A setting method for a machine learning model according to one aspect of the present disclosure includes: detecting an input operation of specifying at least one of a sensitivity or a specificity of a machine learning model for medical diagnosis; determining, based on the at least one of the sensitivity or the specificity specified by the detected input operation, a combination of a value of the sensitivity and a value of the specificity; and setting a cut off value of the machine learning model corresponding to the determined combination.

### BRIEF DESCRIPTION OF DRAWINGS

A more complete understanding of this application can be obtained when the following detailed description is considered in conjunction with the following drawings, in which:

FIG. 1 is a drawing illustrating an example of the configuration of a system according to some embodiments;

FIG. 2 is a flowchart illustrating the flow of cut off value setting processing performed by the system according to some embodiments;

FIG. 3 is a flowchart illustrating the flow of cut off value setting processing performed by the system according to Embodiment 1;

FIG. 4 is an example of a screen displayed by the system according to Embodiment 1;

FIG. 5 is another example of a screen displayed by the system according to Embodiment 1;

FIG. 6 is a flowchart illustrating the flow of cut off value setting processing performed by the system according to Embodiment 2;

FIG. 7 is an example of a screen displayed by the system according to Embodiment 2;

FIG. 8 is another example of a screen displayed by the system according to Embodiment 2;

FIG. 9 is a flowchart illustrating the flow of cut off value setting processing performed by the system according to Embodiment 3;

FIG. 10 is an example of a screen displayed by the system according to Embodiment 3;

FIG. 11 is a flowchart illustrating the flow of cut off value setting processing performed by the system according to Embodiment 4;

FIG. 12 is an example of a screen displayed by the system according to Embodiment 4; and

FIG. 13 is a flowchart illustrating the flow of cut off value setting processing performed by the system according to Embodiment 5.

### DETAILED DESCRIPTION OF THE INVENTION

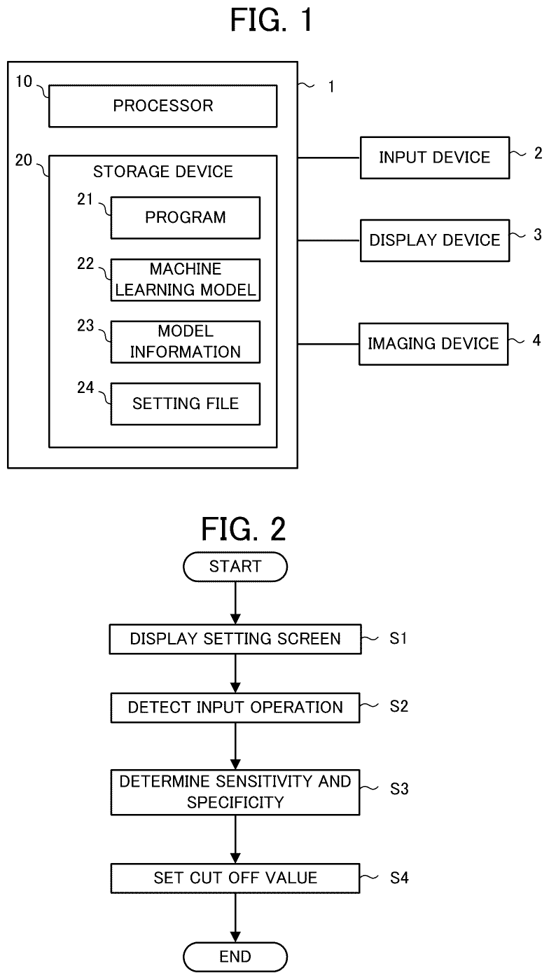

FIG. 1 is a drawing illustrating an example of the configuration of a system according to some embodiments. The system illustrated in FIG. 1 is a diagnosis support device that uses a machine learning model for medical diagnosis, and is configured to determine a positive/negative for a predetermined disease on the basis of an inputted image, and output determination results. Additionally, the diagnosis support device illustrated in FIG. 1 is configured so that a user can set a cut off value for identifying the positive/negative. In the following, the configuration of the diagnosis support device is described while referencing FIG. 1.

The diagnosis support device illustrated in FIG. 1 includes an information processing device 1, an input device 2, a display device 3, and an imaging device 4. The information processing device 1 is a device that includes a processor 10 and a storage device 20, and may be implemented as a general-use personal computer. In one example, the input device 2 is implemented as a keyboard, a touch device, or the like. In one example, the display device 3 is implemented as a liquid crystal display or the like. The imaging device 4 is a device that acquires medical images and, for example, is implemented as a dermoscopy camera or the like.

A program 21, a machine learning model 22, model information 23, a setting file 24, and the like are stored in the storage device 20 of the information processing device 1. The machine learning model 22 is a trained machine learning model for medical diagnosis that outputs determination results (positive or negative) for a predetermined disease in response to an input of a medical image.

The model information 23 includes information expressing performances of the machine learning model 22 for every cut off value, which is a reference for identifying positive and negative. Specifically, the model information 23 includes combinations of a sensitivity and a specificity that are precision indexes, calculated for every cut off value, of the machine learning model 22. That is, the model information 23 includes combinations of the sensitivity, the specificity, and the cut off value.

The setting file 24 is a file in which settings of the diagnosis support device are stored. For example, the cut off value set for the machine learning model 22 is stored in the setting file 24. In addition to the cut off value, display settings of a setting screen, described later, may be stored in the setting file 24. The setting screen is for setting the cut off value of the machine learning model 22.

In the information processing device 1 configured as described above, the processor 10 executes the program 21 to perform determination processing using the machine learning model 22 on the medical image acquired by the imaging device 4, and outputs the determination results that express positive or negative. Additionally, the information processing device 1 executes the program 21 to change the settings of the machine learning model 22 on the basis of an operation of the user performed using the input device 2. Specifically, the user changes the cut off value of the machine learning model 22 to change the balance between the sensitivity and the specificity of the machine learning model 22.

FIG. 2 is a flowchart illustrating the flow of cut off value setting processing performed by the system according to some embodiments. In the following, processing, performed by the information processing device 1, for changing the cut off value in accordance with an operation of a user is described while referencing FIG. 2. Note that the processing illustrated in FIG. 2 is started by the processor 10 executing the program 21.

When the processing illustrated in FIG. 2 starts, firstly, the processor 10 displays, on the display device 3, a setting screen for setting the cut off value of the machine learning model 22 (step S1). The setting screen is a screen on which, instead of directly specifying the cut off value of the machine learning model 22, at least one of the sensitivity and the specificity of the machine learning model 22 can be specified.

Note that the “sensitivity” of the machine learning model 22 is a rate of determinations of positive in a target disease group, that is, a rate (true positive rate) at which the machine learning model 22 correctly determines positive as positive. The “specificity” of the machine learning model 22 is a rate of determinations of negative in a non-disease group, that is, a rate (true negative rate) at which the machine learning model 22 correctly determines negative as negative.

Next, when the user specifies at least one of the sensitivity and the specificity of the machine learning model 22 on the setting screen, the processor 10 detects that input operation (step S2). The sensitivity and the specificity of the trained machine learning model 22 are related such that, when one is set, the other is determined. Using this relationship, the processor 10 determines a combination of a value of the sensitivity and a value of the specificity on the basis of the at least one of the sensitivity and the specificity specified by the detected input operation (step S3).

When the combination of the sensitivity and the specificity is determined, the processor 10 sets, to the machine learning model 22, the cut off value corresponding to that combination (step S4), and ends the cut off value setting processing illustrated in FIG. 2. For example, in step S4, the processor 10 reads out, from the model information 23, the cut off value corresponding to the combination of the sensitivity and the specificity determined in step S3, and sets the read-out cut off value to the machine learning model 22.

The cut off value must be changed in order to change the operation of the machine learning model 22 so as to obtain a desired balance between the sensitivity and the specificity. However, the cut off value is an operating parameter of the machine learning model 22, and it is difficult for the user to intuitively ascertain the values that must be set in order to realize operations whereby the desired performance (in this example, the sensitivity and the specificity) will be exhibited in the machine learning model 22. Accordingly, it is difficult for the user to directly specify the cut off value whereby the desired balance between the sensitivity and the specificity will be obtained.

In contrast, with the information processing device 1, it is sufficient that the user specifies at least one of the sensitivity and the specificity that constituted a combination that has the desired balance, and the user needs not specify the cut off value itself. The information processing device 1 determines, on the basis of the specification operation of the user, the cut off value to be set in order to realize the desired balance between the sensitivity and the specificity, and sets that cut off value to the machine learning model 22. That is, it is sufficient that the user simply and directly specifies the desired performance (the balance between the sensitivity and the specificity) and, on the basis of that specification, the information processing device 1 can set the required cut off value to the machine learning model 22.

Application examples of the cut off value setting method described above include cases of changing the cut off value in accordance with the use scene. For example, in the case of a screening test, a setting is desirable in which the sensitivity is increased to prioritize avoiding overlooking lesions. Meanwhile, in the case of investigating a site to be removed in surgery, it is necessary to avoid mistakenly cutting parts that are not lesions and, as such, a setting is desirable in which the specificity is higher than in the screening test described above. Thus, the optimal balance between the sensitivity and the specificity changes in accordance with the use scene but, according to the information processing device 1 of the present embodiment, the user can directly specify the sensitivity and the specificity and, as such, it is possible to easily realize a setting of an optimal cut off value that corresponds to the use scene. Therefore, according to the information processing device 1, diagnosis support can be performed at an optimal setting that corresponds to the use scene.

In the following, specific examples are described of methods in which the performance of the machine learning model 22 is specified and, as a result, the information processing device 1 sets the cut off value whereby that performance is exhibited.

### Embodiment 1

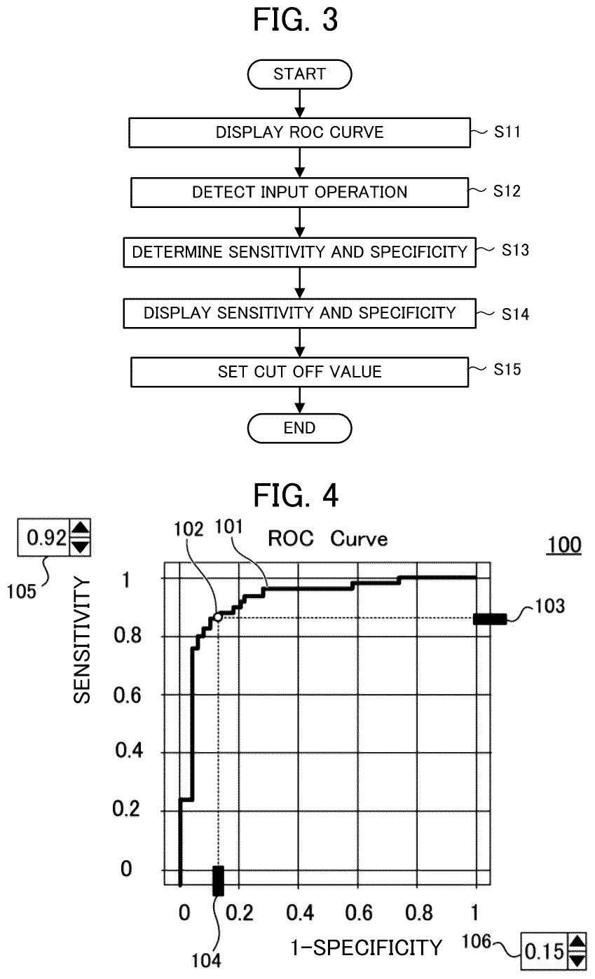

FIG. 3 is a flowchart illustrating the flow of the cut off value setting processing performed by the system according to present embodiment. FIG. 4 is an example of a screen displayed by the system according to the present embodiment.

The present embodiment is an example in which a receiver operating characteristic (ROC) curve, that expresses the relationship between the sensitivity and the specificity, is displayed on a setting screen. Note that the configuration of the system according to the present embodiment is the same as the system (the diagnosis support device) illustrated in FIG. 1.

In the system according to the present embodiment, the cut off value setting processing illustrated in FIG. 3 is performed.

The processing illustrated in FIG. 3 is started by the processor 10 executing the program 21. When the processing illustrated in FIG. 3 starts, firstly, the processor 10 displays, on the display device 3, a setting screen 100 illustrated in FIG. 4 for setting the cut off value of the machine learning model 22 and, in the setting screen 100, displays a graph 101 expressing the relationship between the sensitivity and the specificity of the machine learning model 22 on the display device 3 (step S11).

The graph 101 illustrated in FIG. 4 is a ROC curve in which the sensitivity and the specificity are expressed on the two axes. More specifically, the sensitivity is expressed on the vertical axis and 1—the specificity is expressed on the horizontal axis. The graph 101 is displayed on the basis of information, about the combination of the sensitivity and the specificity for every cut off value, that is stored in the storage device 20 as the model information 23 and that is calculated in advance for the machine learning model 22.

Then, when the user specifies the sensitivity and the specificity of the machine learning model 22 on the setting screen 100, the processor 10 detects that input operation (step S12). As illustrated in FIG. 4, a slider 103 and a slider 104 that slide on each of the axes, that express the sensitivity and the specificity of the graph 101, are provided in the setting screen 100 The user can specify the sensitivity and the specificity by roughly moving a point 102 on the graph 101 by operating the slider 103 or the slider 104 provided in the setting screen 100. Note that the slider 103 and the slider 104 are configured such that, when one is moved, the other moves in conjunction therewith.

Additionally, a spin button 105 for fine-adjusting the sensitivity, and a spin button 106 for fine-adjusting the specificity are provided in the setting screen 100. In step S12, the user can specify the sensitivity and the specificity by finely moving the point 102 on the graph 101 by operating the spin button 105 or the spin button 106 provided in the setting screen 100. Note that the spin button 105 and the spin button 106 are configured so that when one changes, the other changes in conjunction therewith.

Specifically, the user can, by operating the sliders or the spin buttons in the setting screen 100, move the point 102 expressing the sensitivity and the specificity set to the machine learning model 22 between a plurality of points on the graph 101 that correspond to a plurality of combinations of the sensitivity and the specificity stored in the model information 23. These operations are an example of the operation of specifying the sensitivity and the specificity for the region in which the graph 101 is displayed.

The processor 10 determines, on the basis of the input operation detected in step S12 and as the combination of the sensitivity and the specificity desired by the user, the sensitivity and the specificity corresponding to the point 102 (step S13). At this time, the determined sensitivity and specificity are displayed on the spin buttons. That is, in the cut off value setting processing illustrated in FIG. 3, processing is performed for displaying, on the display device 3, the sensitivity and the specificity determined in step S13.

When the combination of the sensitivity and the specificity is determined, the processor 10 sets the cut off value corresponding to that combination to the machine learning model 22 (step S14), and ends the cut off value setting processing illustrated in FIG. 3. The processing of step S14 is the same as the processing of step S4 illustrated in FIG. 2.

Thus, in the cut off value setting method illustrated in FIG. 3, the cut off value of the machine learning model 22 can be set, on the basis of an operation by the user of specifying the sensitivity and the specificity, so that the sensitivity and the specificity have the desired balance. In particular, in the present embodiment, the ROC curve is displayed on the setting screen 100 and, as such, the system can cause the user to ascertain the relationship between the sensitivity and the specificity unique to the machine learning model 22. As a result, the user can easily perform settings according to the use scene. As a result, according to the present embodiment, it is possible to operate the machine learning model 22 at optimal settings. Moreover, in the present embodiment, the sensitivity and the specificity specified on the graph are displayed on the display device 3 and, as such, the user can accurately ascertain the specified sensitivity and specificity.

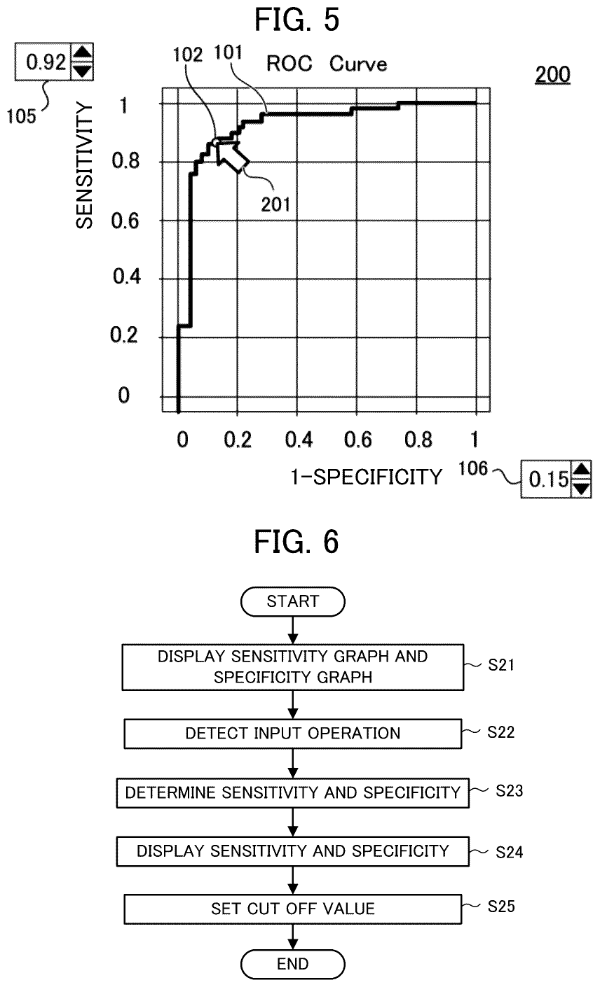

FIG. 5 is another example of a screen displayed by the system according to the present embodiment. In FIG. 4, an example is illustrated in which the point 102 is roughly moved by the sliders provided in the setting screen 100 and approximate sensitivity and specificity are specified, but the method of specifying the sensitivity and the specificity is not limited to this method. As illustrated in FIG. 5, a configuration is possible in which the sensitivity and the specificity are specified by specifying a point on the graph 101 directly using a cursor 201. Note that the feature of adjusting, by spin buttons (the spin button 105 and the spin button 106) the sensitivity and the specificity specified by the cursor 201 is the same as in the example illustrated in FIG. 4.

In the cut off value setting method illustrated in FIG. 3, even when a setting screen 200 illustrated in FIG. 5 is displayed, the same effects can be obtained as when the setting screen 100 illustrated in FIG. 4 is displayed.

### Embodiment 2

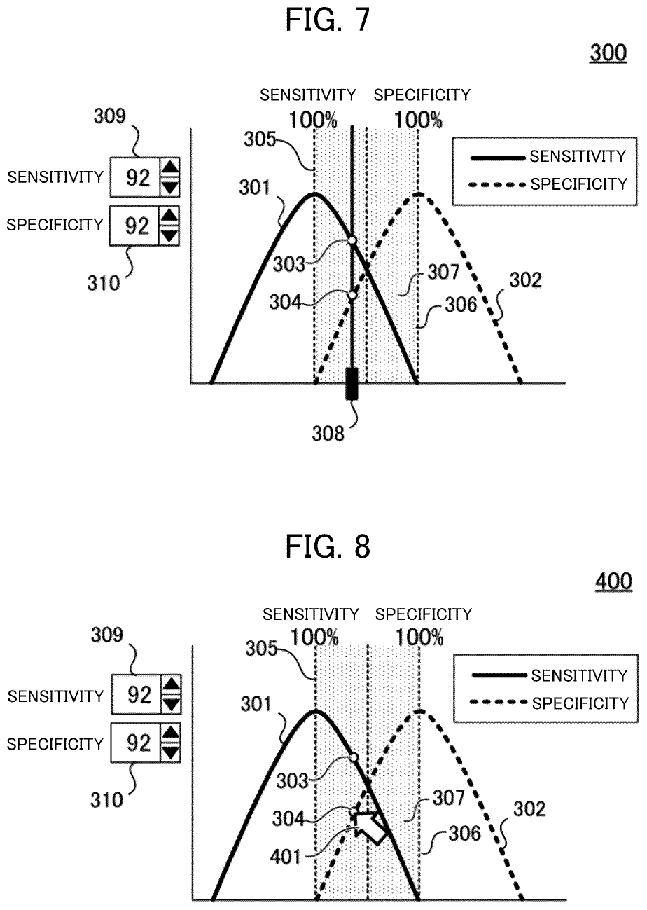

FIG. 6 is a flowchart illustrating the flow of the cut off value setting processing performed by the system according to present embodiment. FIG. 7 is an example of a screen displayed by the system according to the present embodiment.

The present embodiment is an example in which a graph expressing changes in the sensitivity and a graph expressing changes in the specificity are displayed on a setting screen. Note that the configuration of the system according to the present embodiment is the same as the system (the diagnosis support device) illustrated in FIG. 1. In the system according to the present embodiment, the cut off value setting processing illustrated in FIG. 6 is performed.

The processing illustrated in FIG. 6 is started by the processor 10 executing the program 21. When the processing illustrated in FIG. 6 starts, firstly, the processor 10 displays, on the display device 3, a setting screen 300 illustrated in FIG. 7 for setting the cut off value of the machine learning model 22 and, in the setting screen 300, displays, on the display device 3, a graph 301 (sensitivity graph) that expresses changes in the sensitivity relative to the setting of the machine learning model 22, and a graph 302 (specificity graph) that expresses changes in the specificity relative to the setting of the machine learning model 22 (step S21).

The graph 301 illustrated in FIG. 7 is a graph in which the setting is expressed on the horizontal axis and the sensitivity is expressed on the vertical axis. The graph 302 illustrated in FIG. 7 is a graph in which the setting is expressed on the horizontal axis and the specificity is expressed on the vertical axis. The settings expressed by these graphs correspond with the cut off values on a one-to-one basis. The axis that expresses the setting is shared by the graph 301 and the graph 302 and, as such, the sensitivity and the specificity corresponding to the same setting are displayed side by side on a single straight line in the vertical axis direction. Note that the graph 301 and the graph 302 are displayed on the basis of the information, about the combination of the sensitivity and the specificity for every cut off value, that is stored in the storage device 20 as the model information 23 and that is calculated in advance for the machine learning model 22.

Then, when the user specifies the sensitivity and the specificity of the machine learning model 22 on the setting screen 300, the processor 10 detects that input operation (step S22). As illustrated in FIG. 7, a slider 308 that slides on the axis that expresses the setting is provided in the setting screen 300.

A movement range of the slider 308 corresponds to a region 307 between a setting (line 305) corresponding to 100% sensitivity expressed by the peak of the graph 301 and a setting (line 306) corresponding to 100% specificity expressed by the peak of the graph 302. The user can specify the sensitivity and the specificity by roughly moving a point 303 on the graph 301 that expresses the sensitivity and a point 304 on the graph 302 that expresses the specificity corresponding to the position of the slider 308 by operating the slider 308 provided in the setting screen 300.

Additionally, the setting screen 300 includes a spin button 309 for fine-adjusting the sensitivity, and a spin button 310 for fine-adjusting the specificity. In step S22, the user can specify the sensitivity and the specificity by finely moving the point 303 and the point 304 on the graph 301 and the graph 302 by operating the spin button 309 or the spin button 310 provided in the setting screen 300.

The processor 10 determines, on the basis of the input operation detected in step S22 and as the combination of the sensitivity and the specificity desired by the user, the sensitivity corresponding to the point 303 and the specificity corresponding to the point 304 (step S23). At this time, the determined sensitivity and specificity are displayed on the spin buttons. That is, in the cut off value setting processing illustrated in FIG. 6, processing is performed for displaying, on the display device 3, the sensitivity and the specificity determined in step S23.

When the combination of the sensitivity and the specificity is determined, the processor 10 sets the cut off value corresponding to that combination to the machine learning model 22 (step S24), and ends the cut off value setting processing illustrated in FIG. 6. The processing of step S24 is the same as the processing of step S4 illustrated in FIG. 2.

Thus, as in the cut off value setting method illustrated in FIG. 3, in the cut off value setting method illustrated in FIG. 6 as well, the cut off value of the machine learning model 22 can be set, on the basis of an operation by the user of specifying the sensitivity and the specificity, so that the sensitivity and the specificity have the desired balance. Additionally, in the present embodiment, the two graphs respectively related to the sensitivity and the specificity are displayed instead of the ROC curve, and these two graphs also are graphs that express the relationship of the sensitivity and the specificity of the machine learning model 22. Specifically, the present embodiment is the same as Embodiment 1 in that graphs expressing the relationship between the sensitivity and the specificity of the machine learning model 22 are displayed on the display device 3, and the sensitivity and the specificity corresponding to the cut off value to be set are determined by the operation of specifying the sensitivity and the specificity for the region in which the graphs are displayed. As such, the present embodiment is the same as Embodiment 1 in that the user can ascertain, by the graphs, the relationship between the sensitivity and the specificity unique to the machine learning model 22, and the user can easily perform settings according to the use scene. Thus, according to the present embodiment as well, it is possible to operate the machine learning model 22 at optimal settings.

FIG. 8 is another example of a screen displayed by the system according to the present embodiment. In FIG. 7, an example is illustrated in which the point 303 and the point 304 are roughly moved by the slider provided in the setting screen 300 and approximate sensitivity and specificity are specified, but the method of specifying the sensitivity and the specificity is not limited to this method. As illustrated in FIG. 8, a configuration is possible in which the sensitivity and the specificity are specified by directly specifying, using a cursor 401, a point on the graph 301 or the graph 302 displayed on a setting screen 400. Note that the feature of adjusting, by spin buttons (the spin button 309 and the spin button 310) the sensitivity and the specificity specified by the cursor 401 is the same as in the example illustrated in FIG. 7.

### Embodiment 3

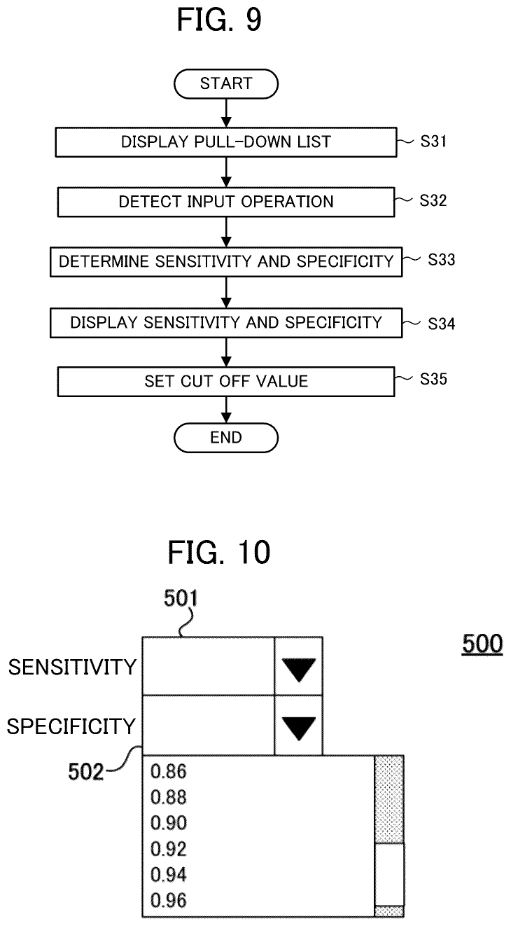

FIG. 9 is a flowchart illustrating the flow of the cut off value setting processing performed by the system according to present embodiment. FIG. 10 is an example of a screen displayed by the system according to the present embodiment. The present embodiment is an example in which one of the sensitivity and the specificity is specified from among options displayed in a pull-down list 501. Note that the configuration of the system according to the present embodiment is the same as the system (the diagnosis support device) illustrated in FIG. 1. In the system according to the present embodiment, the cut off value setting processing illustrated in FIG. 9 is performed.

The processing illustrated in FIG. 9 is started by the processor 10 executing the program 21. When the processing illustrated in FIG. 9 starts, firstly, the processor 10 displays, on the display device 3, a setting screen 500 illustrated in FIG. 10 for setting the cut off value of the machine learning model 22, and displays the pull-down list 501 and a pull-down list 502 in the setting screen 500 (step S31).

Note that the pull-down list 501 is a pull-down list for selecting the sensitivity from among a plurality of options. The plurality of options included in the pull-down list 501 is a plurality of sensitivities, that form combinations of the sensitivity and the specificity for every cut off value, that are stored in the storage device 20 as the model information 23. Additionally, the pull-down list 502 is a pull-down list for selecting the specificity from among a plurality of options, and this plurality of options is a plurality of sensitivities, that form combinations of the sensitivity and the specificity for every cut off value, that are stored in the storage device 20 as the model information 23.

Then, when the user specifies the sensitivity and the specificity of the machine learning model 22 on the setting screen 500, the processor 10 detects that input operation (step S32) and determines the sensitivity and the specificity (step S33).

For example, when the user operates the pull-down list 501 and selects the sensitivity, the specificity corresponding to that sensitivity selected from the pull-down list 501 is automatically selected from pull-down list 502 and, as a result, the combination of the sensitivity and the specificity is determined. Additionally, when the user operates the pull-down list 502 and selects the specificity, the sensitivity corresponding to that specificity selected from the pull-down list 502 is automatically selected from pull-down list 501 and, as a result, the combination of the sensitivity and the specificity is determined. Thus, the determined sensitivity and specificity are displayed in pull-down lists. That is, in the cut off value setting processing illustrated in FIG. 9, processing is performed for displaying, on the display device 3, the sensitivity and the specificity determined in step S33.

When the combination of the sensitivity and the specificity is determined, the processor 10 sets the cut off value corresponding to that combination to the machine learning model 22 (step S34), and ends the cut off value setting processing illustrated in FIG. 9. The processing of step S34 is the same as the processing of step S4 illustrated in FIG. 2.

Thus, as in the cut off value setting method illustrated in FIG. 3, in the cut off value setting method illustrated in FIG. 9 as well, the cut off value of the machine learning model 22 can be set, on the basis of an operation by the user of specifying the sensitivity and the specificity, so that the sensitivity and the specificity have the desired balance.

### Embodiment 4

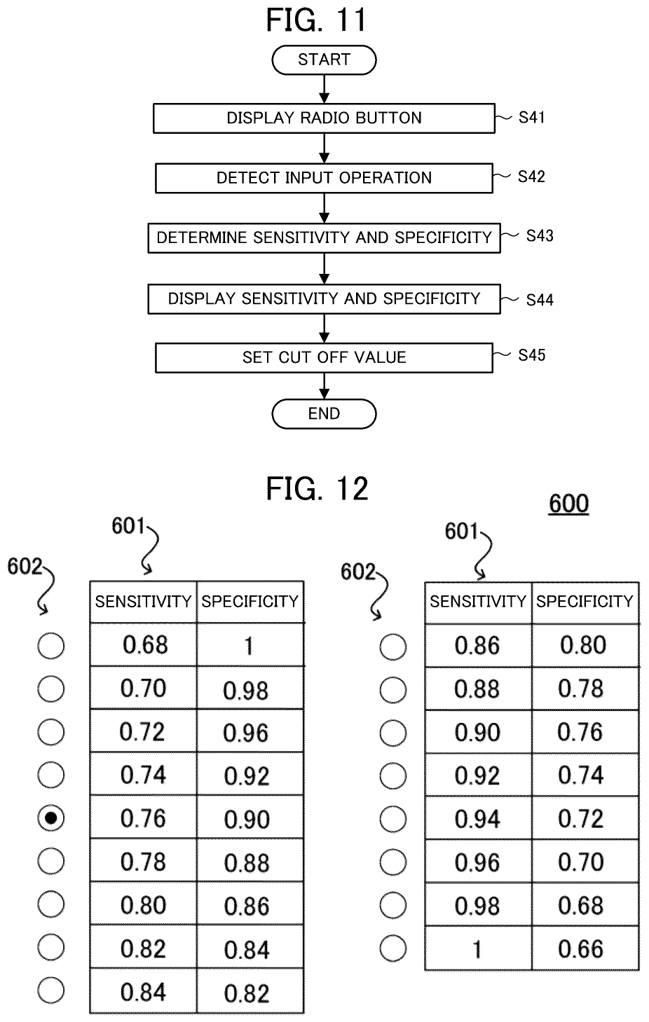

FIG. 11 is a flowchart illustrating the flow of the cut off value setting processing performed by the system according to present embodiment. FIG. 12 is an example of a screen displayed by the system according to the present embodiment. The present embodiment is an example in which the combination of the sensitivity and the specificity is specified from among options for the combination of the sensitivity and the specificity displayed in a table format. Note that the configuration of the system according to the present embodiment is the same as the system (the diagnosis support device) illustrated in FIG. 1. In the system according to the present embodiment, the cut off value setting processing illustrated in FIG. 11 is performed.

The processing illustrated in FIG. 11 is started by the processor 10 executing the program 21.

When the processing illustrated in FIG. 11 starts, firstly, the processor 10 displays, on the display device 3, a setting screen 600 illustrated in FIG. 12 for setting the cut off value of the machine learning model 22, and displays, in the setting screen 600, a table 601 expressing a plurality of combinations of the sensitivity and the specificity, and a radio button 602 for selecting one combination among the plurality of combinations of expressed in the table 601 (step S41). Note that the plurality of combinations included in the table 601 are combinations of the sensitivity and the specificity for every cut off value that is stored in the storage device 20 as the model information 23.

Then, when the user specifies the sensitivity and the specificity of the machine learning model 22 on the setting screen 600, the processor 10 detects that input operation (step S42) and determines the sensitivity and the specificity (step S43). Specifically, the sensitivity and the specificity are determined by the user operating the radio button 602 and specifying any one combination among the plurality of combinations included in the table 601.

When the combination of the sensitivity and the specificity is determined, the processor 10 sets the cut off value corresponding to that combination to the machine learning model 22 (step S44), and ends the cut off value setting processing illustrated in FIG. 11. The processing of step S44 is the same as the processing of step S4 illustrated in FIG. 2.

Thus, as in the cut off value setting method illustrated in FIG. 3, in the cut off value setting method illustrated in FIG. 11 as well, the cut off value of the machine learning model 22 can be set, on the basis of an operation by the user of specifying the sensitivity and the specificity, so that the sensitivity and the specificity have the desired balance. Additionally, in the present embodiment, the plurality of combinations of the sensitivity and the specificity are displayed in the table 601 as a single list and, as such, the user can ascertain the relationship between the sensitivity and the specificity unique to the machine learning model 22, and the user can easily perform settings according to the use scene. Thus, according to the present embodiment as well, it is possible to operate the machine learning model 22 at optimal settings.

### Embodiment 5

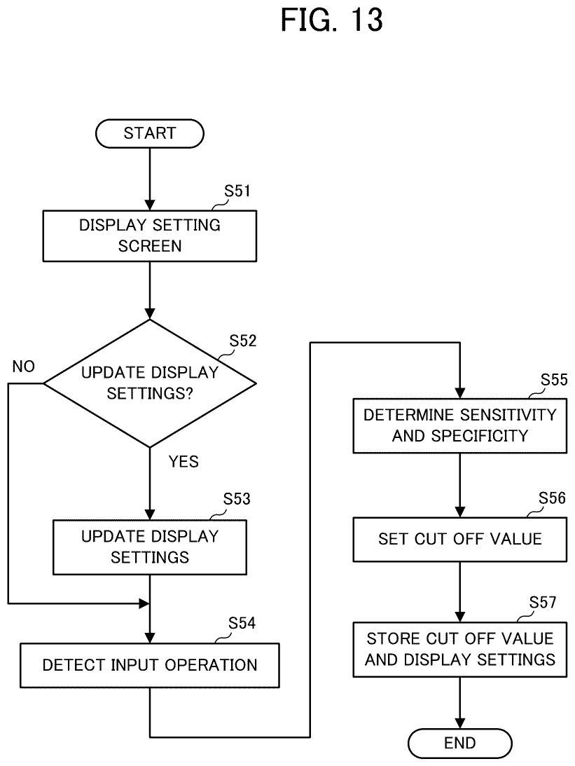

FIG. 13 is a flowchart illustrating the flow of the cut off value setting processing performed by the system according to present embodiment. The present embodiment is an example in which the user can freely set display settings of the setting screen, and the display settings of the setting screen are stored together with the set cut off value. Note that the configuration of the system according to the present embodiment is the same as the system (the diagnosis support device) illustrated in FIG. 1. In the system according to the present embodiment, the cut off value setting processing illustrated in FIG. 13 is performed.

The processing illustrated in FIG. 13 is started by the processor 10 executing the program 21.

When the processing illustrated in FIG. 13 starts, firstly, the processor 10 displays, on the display device 3, a setting screen for setting the cut off value of the machine learning model 22 (step S51). Note that the setting screen displayed in step S51 is, for example, any one of the setting screen 100 to the setting screen 600 described above.

Then, the processor 10 determines whether an update operation for the display settings of the setting screen has been performed (step S52). Note that the display settings of the setting screen includes a display setting related to a specification method of the sensitivity and the specificity, a setting related to a numerical value display of the sensitivity and the specificity, a setting related to the cut off value display, and the like.

The display settings related to the specification method of the sensitivity and the specificity include, for example, a setting for displaying the ROC curve illustrated in FIG. 4 or 5, a setting for displaying the sensitivity graph and the specificity graph illustrated in FIG. 7 or 8, a setting for displaying the pull-down list illustrated in FIG. 10, a setting for displaying the table and the radio button illustrated in FIG. 12, and the like. The user may select any one of these settings, or may select and set a plurality of these settings.

The setting related to the numerical value display of the sensitivity and the specificity is a setting related to the numerical value display of the sensitivity and the specificity displayed on the spin buttons illustrated in FIG. 4, 5, 7, or 8, the pull-down list illustrated in FIG. 10, or the table illustrated in FIG. 11, and includes, for example, a ratio/percentage display setting, a display setting for the number of significant digits, and the like.

The setting related to the cut off value display includes a setting for whether to display the cut off value corresponding to the specified sensitivity and specificity, that is, whether to display the set cut off value. Furthermore, the setting related to the cut off value display includes a display format setting for the cut off value when displaying, examples of such being a display setting for the number of significant digits, and the like.

In step S52, when a determination is made that an update operation of the display settings has been performed, the processor 10 updates the display settings to the changed settings (step S53). Then, when the user specifies the sensitivity and the specificity of the machine learning model 22 on the setting screen, the processor 10 detects that input operation (step S54), determines the sensitivity and the specificity (step S55), and sets the cut off value (step S56). The processing of steps S54 to S56 is the same as the corresponding processing in the cut off value setting processing according to the embodiments described above.

Finally, the processor 10 associates the display settings of the setting screen updated in step S53 with the cut off value set in step S56 and stores the associated information in the setting file 24 (step S57), and ends the cut off value setting processing illustrated in FIG. 13.

Thus, as in the cut off value setting method illustrated in FIG. 3, in the cut off value setting method illustrated in FIG. 13 as well, the cut off value of the machine learning model 22 can be set, on the basis of an operation by the user of specifying the sensitivity and the specificity, so that the sensitivity and the specificity have the desired balance. Additionally, in the present embodiment, it is possible to set the display settings of the setting screen and, as such, the user can freely select the setting method of the cut off value. Furthermore, the appropriately changed display settings are stored together with the cut off value in the setting file and, as such, when next displaying the display screen, the setting screen can be automatically displayed with the preferred display settings, without readjusting the display settings.

The embodiments described above are given as specific examples for the purpose of facilitating comprehension of the present disclosure. However, it should be understood that the present disclosure is not limited to the embodiments, and that various modifications and alternatives to the embodiments are included in the scope of the present disclosure. For example, is should be understood that the constituents of the embodiments described above can be modified without departing from the spirit and scope of the embodiments. Additionally, it should be understood that the plurality of constituents disclosed in the embodiments described above can be appropriately combined to realize various embodiments. Furthermore, a person skilled in the art would understand that some of the constituents may be omitted from the entirety of the constituents described in the embodiments, or some constituents may be added to the constituents described in the embodiments to realize various embodiments. That is, the image processing device, the image processing method, and the program described above can be modified and changed without departing from the spirit and scope of the claims.

In the embodiments described above, an example is described in which the spin buttons are displayed together with a graph on the setting screen, but a configuration is possible in which the displaying of the spin buttons is omitted. Additionally, a configuration is possible in which, instead of the spin buttons, a pull-down list such as illustrated in FIG. 10 or a combination of a table and a radio button such as illustrated in FIG. 12 is displayed.

In the embodiments described above, the target to be identified by the machine learning model 22 is not particularly limited, but an example thereof is a tumor. A configuration is possible in which the machine learning model 22 is a model that outputs, as positive or negative, whether an area in a medical image is a tumor or is not a tumor.

In the embodiments described above, an example is described in which the diagnosis support device includes the imaging device 4, but a configuration is possible in which the imaging device 4 is not included. A configuration is possible in which the diagnosis support device is an image processing device that analyzes medical images acquired by a different device. Additionally, a configuration is possible in which the functions of the input device, the functions of the display device, the functions of the imaging device, and the plurality of functions corresponding to each of the plurality of processings executed by the diagnosis support device are divided among and executed by a plurality of devices. For example, the various functions (various processings) may be divided, in desired combinations, among and executed by a system including a server and a terminal device connected by a network.

The foregoing describes some example embodiments for explanatory purposes. Although the foregoing discussion has presented specific embodiments, persons skilled in the art will recognize that changes may be made in form and detail without departing from the broader spirit and scope of the invention. Accordingly, the specification and drawings are to be regarded in an illustrative rather than a restrictive sense. This detailed description, therefore, is not to be taken in a limiting sense, and the scope of the invention is defined only by the included claims, along with the full range of equivalents to which such claims are entitled.

1. A diagnosis support system that executes:
processing for detecting an input operation of specifying at least one of a sensitivity or a specificity of a machine learning model for medical diagnosis;
processing for determining, based on the at least one of the sensitivity or the specificity specified by the detected input operation, a combination of a value of the sensitivity and a value of the specificity; and
processing for setting a cut off value of the machine learning model corresponding to the determined combination.

2. The diagnosis support system according to claim 1, wherein
the machine learning model is a trained model trained so as to determine, based on inputted information, a positive/negative for a predetermined disease,
the cut off value is a reference for identifying the positive/negative,
the sensitivity is a rate at which the positive is correctly determined as positive,
the specificity is a rate at which the negative is correctly determined as negative, and
the machine learning model is trained such that the sensitivity and the specificity differ for every of the cut off value.

3. The diagnosis support system according to claim 2, further executing processing for:
storing model information including information that expresses a performance of the machine learning model, the information expressing the sensitivity and the specificity for each of a plurality of the cut off value that differ from each other, and
determining, based on the model information, the cut off value for the combination of a value of the sensitivity and a value of the specificity.

4. The diagnosis support system according to claim 1, further executing processing for:
displaying, on a display device, a graph expressing a relationship between the sensitivity and the specificity of the machine learning model, wherein
the input operation is an operation of specifying the sensitivity and the specificity for a region in which the graph is displayed.

5. The diagnosis support system according to claim 4, wherein
the graph is a ROC graph having the sensitivity and the specificity on two axes.

6. The diagnosis support system according to claim 4, wherein
the graph includes
a sensitivity graph expressing a change of the sensitivity relative to a setting of the machine learning model, and
a specificity graph expressing a change of the specificity relative to the setting of the machine learning model, and
the sensitivity graph and the specificity graph share an axis that expresses the setting of the machine learning model.

7. The diagnosis support system according to claim 1, further executing processing for:
displaying, on a display device, the sensitivity and the specificity of the determined combination.

8. The diagnosis support system according to claim 1, further executing processing for:
associating and storing a display setting of a setting screen for setting the cut off value of the machine learning model, and the set cut off value.

9. The diagnosis support system according to claim 1, wherein
the machine learning model is a trained model trained so as to determine, based on an inputted image, a positive/negative of a disease related to a tumor.

10. The diagnosis support system according to claim 1, further comprising:
a controller that executes various processings of the diagnosis support system; an inputter that performs an input operation of specifying at least one of the sensitivity or the specificity; a display that displays information including the sensitivity and the specificity; and an imager that captures an image to be input into the machine learning model.

11. A setting method for a machine learning model, comprising:
detecting an input operation of specifying at least one of a sensitivity or a specificity of a machine learning model for medical diagnosis;
determining, based on the at least one of the sensitivity or the specificity specified by the detected input operation, a combination of a value of the sensitivity and a value of the specificity; and
setting a cut off value of the machine learning model corresponding to the determined combination.

12. A non-transitory computer readable recording medium storing a program that causes a computer to execute:
processing for detecting an input operation of specifying at least one of a sensitivity or a specificity of a machine learning model for medical diagnosis;
processing for determining, based on the at least one of the sensitivity or the specificity specified by the detected input operation, a combination of a value of the sensitivity and a value of the specificity; and
processing for setting a cut off value of the machine learning model corresponding to the determined combination.
